(19) United States
(12) Patent Application Publication
(10) Pub. No.: US 2025/0367506 A1
(43) Pub. Date: Dec. 4, 2025

(54) SYSTEM AND METHOD FOR ANALYSIS OF SPORTS BIOMECHANICS

(71) Applicant: TruRep, LLC, Mt. Pleasant, SC (US)

(72) Inventor: Wesley Horne, Mt. Pleasant, SC (US)

(21) Appl. No.: 19/207,686

(22) Filed: May 14, 2025

Related U.S. Application Data
(60) Provisional application No. 63/647,367, filed on May 14, 2024.

(51) Int. Cl. A63B 24/00

(52) U.S. Cl. CPC A63B 24/0062; A63B 24/0003; A63B 2220/836; A63B 2225/50

(57) ABSTRACT

A system and method for analyzing a user's biomechanics for one or more sports through the use of one or more wearable sensor devices. Aspects of the present disclosure provide for a system and method for tracking the biomechanical movements of a user via one or more body-worn sensor devices. Data from the one or more body-worn sensor devices is communicated to one or more processor and analyzed according to one or more algorithms configured to analyze one or more variables associated with a specified biomechanical movement. An end user application is configured to generate one or more feedback outputs in order to improve the user's biomechanical technique.

### CROSS-REFERENCE TO RELATED APPLICATIONS

The present application claims priority benefit of U.S. Provisional Application Ser. No. 63/647,367 filed May 14, 2024, entitled “SYSTEM AND METHOD FOR ANALYSIS OF SPORTS BIOMECHANICS”; the entirety of which is hereby incorporated by reference herein.

### FIELD

The present disclosure relates to the field of computerized sports training aids; in particular, a system and method for analyzing a user's biomechanics for one or more sport techniques through the use of one or more wearable sensor devices.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Certain aspects of the present disclosure provide for a system and method for training sports biomechanics comprising providing a wireless communications interface between a wearable sensor device and at least one computing device, wherein the wearable sensor device is worn by a user; launching an instance of an end user application on the at least one computing device; selecting, via a user interface of the end user application, at least one operational mode for the end user application, wherein the at least one operational mode is associated with a specified biomechanical action of the user, wherein the specified biomechanical action is associated with at least one sport technique (e.g., swinging a golfclub), receiving, with the wearable sensor device, one or more sensor inputs in response to the user performing one or more biomechanical actions; processing, according to one or more operations of the end user application, the one or more sensor inputs; and providing, with the wearable sensor device or the at least one computing device, one or more real-time feedback outputs to the user in response to processing the one or more sensor inputs.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 is an illustrative embodiment of a computing device through which one or more aspects of the present disclosure may be implemented;

FIG. 2 is an architecture diagram of a sports biomechanics training system, in accordance with certain aspects of the present disclosure;

FIG. 3 is a functional diagram illustrating certain biomechanics of shooting a basketball;

FIG. 4 is a functional diagram illustrating certain biomechanics of throwing a baseball;

FIG. 5 is a functional diagram illustrating certain biomechanics of swinging a golfclub;

FIG. 6 is a process flow diagram of a routine for a sports biomechanics training method, in accordance with certain aspects of the present disclosure;

FIG. 7 is a process flow diagram of a routine for a sports biomechanics training method, in accordance with certain aspects of the present disclosure;

FIG. 8 is a process flow diagram of a routine of a sports biomechanics training method, in accordance with certain aspects of the present disclosure;

FIG. 9 is a process flow diagram of a routine of a sports biomechanics training method, in accordance with certain aspects of the present disclosure; and

FIG. 10 is a process flow diagram of a routine of a sports biomechanics training method, in accordance with certain aspects of the present disclosure.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatus and systems for sports biomechanics training comprising one or more wearable sensor devices communicably engaged with a mobile computing application configured to measure the real-time movements of a user via one or more sensor inputs and provide real-time analysis and feedback of the user's biomechanics for one or more sports.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Before the present invention and specific exemplary embodiments of the invention are described, it is to be understood that this invention is not limited to the particular embodiments described, and as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, “exemplary” means serving as an example or illustration and does not necessarily denote ideal or best.

As used herein, the term “includes” means includes but is not limited to, the term “including” means including but not limited to. The term “based on” means based at least in part on.

As used herein, the term “interface” refers to any shared boundary across which two or more separate components of a computer system may exchange information. The exchange can be between software, computer hardware, peripheral devices, humans, and combinations thereof.

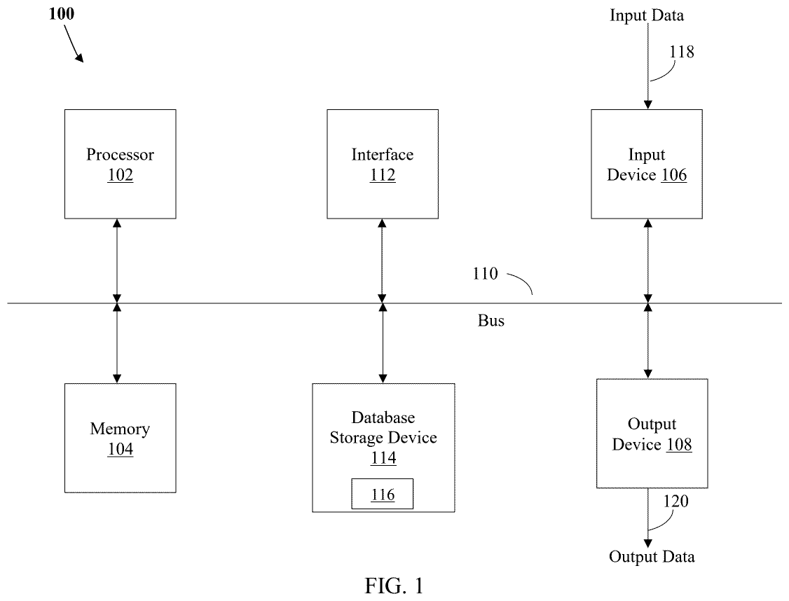

Turning now descriptively to the drawings, in which similar reference characters denote similar elements throughout the several views, FIG. 1 depicts an exemplary computing system in which certain illustrated embodiments of the present invention may be implemented.

Referring now to FIG. 1, a processor-implemented computing system 100 through which one or more aspects of the present disclosure may be implemented is shown. According to an embodiment, system 100 may generally comprise at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could comprise more than one distinct processing device, for example to handle different functions within system 100. Input device 106 receives input data 118 and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can comprise, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, system 100 is adapted to allow data or information to be stored in and/or retrieved from, via wired or wireless communication means, at least one database 116. The interface 112 may allow wired and/or wireless communication between the processing unit 102 and peripheral components that may serve a specialized purpose. In general, the processor 102 can receive instructions as input data 118 via input device 106 and can display processed results or other output to a user by utilizing output device 108. More than one input device 106 and/or output device 108 can be provided. It should be appreciated that system 100 may be any form of terminal, server, specialized hardware, or the like.

It is to be appreciated that system 100 may be a part of a networked communications system. System 100 could connect to a network, for example the Internet or a WAN. Input data 118 and output data 120 could be communicated to other devices via the network. The transfer of information and/or data over the network can be achieved using wired communications means or wireless communications means. A server can facilitate the transfer of data between the network and one or more databases. A server and one or more databases provide an example of an information source.

Thus, system 100 illustrated in FIG. 1 may operate in a networked environment using logical connections to one or more remote computers. The remote computer may be a personal computer, a server, a router, a network PC, a peer device, or other common network node, and typically includes many or all of the elements described above.

It is to be further appreciated that the logical connections depicted in FIG. 1 include a local area network (LAN) and a wide area network (WAN) but may also include other networks such as a personal area network (PAN). Such networking environments are commonplace in offices, enterprise-wide computer networks, intranets, and the Internet. For instance, when used in a LAN networking environment, system 100 is connected to the LAN through a network interface or adapter. When used in a WAN networking environment, the computing system environment typically includes a modem or other means for establishing communications over the WAN, such as the Internet. The modem, which may be internal or external, may be connected to a system bus via a user input interface, or via another appropriate mechanism. In a networked environment, program modules depicted relative to system 100, or portions thereof, may be stored in a remote memory storage device. It is to be appreciated that the illustrated network connections of FIG. 1 are exemplary and other means of establishing a communications link between multiple computers may be used.

FIG. 1 is intended to provide a brief, general description of an illustrative and/or suitable exemplary environment in which embodiments of the present disclosure may be implemented. FIG. 1 is an example of a suitable environment and is not intended to suggest any limitation as to the structure, scope of use, or functionality of an embodiment of the present invention. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

In the description that follows, certain embodiments may be described with reference to acts and symbolic representations of operations that are performed by one or more computing devices, such as system 100 of FIG. 1. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of well-known computing systems, environments, and configurations that may be suitable for use with an embodiment include, but are not limited to, personal computers, handheld or laptop devices, personal digital assistants, multiprocessor systems, microprocessor-based systems, set top boxes, programmable consumer electronics, network, minicomputers, server computers, game server computers, web server computers, mainframe computers, and distributed computing environments that include any of the above systems or devices.

Embodiments may be described in a general context of computer-executable instructions, such as program modules, being executed by a computer. Generally, program modules include routines, programs, objects, components, data structures, etc., that perform particular tasks or implement particular abstract data types. An embodiment may also be practiced in a distributed computing environment where tasks are performed by remote processing devices that are linked through a communications network. In a distributed computing environment, program modules may be located in both local and remote computer storage media including memory storage devices.

With the exemplary computing system environment 100 of FIG. 1 being generally shown and discussed above, description will now turn towards illustrated embodiments of the present invention which generally relates to system and methods for training sports biomechanics through the use of one or more wearable sensor devices communicably engaged with a mobile computing device comprising an end user application configured to analyze one or more sensor inputs and provide real-time analysis and feedback on a user's biomechanics for one or more sport techniques (e.g., swinging a golfclub, throwing a baseball, shooting a basketball, etc.).

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

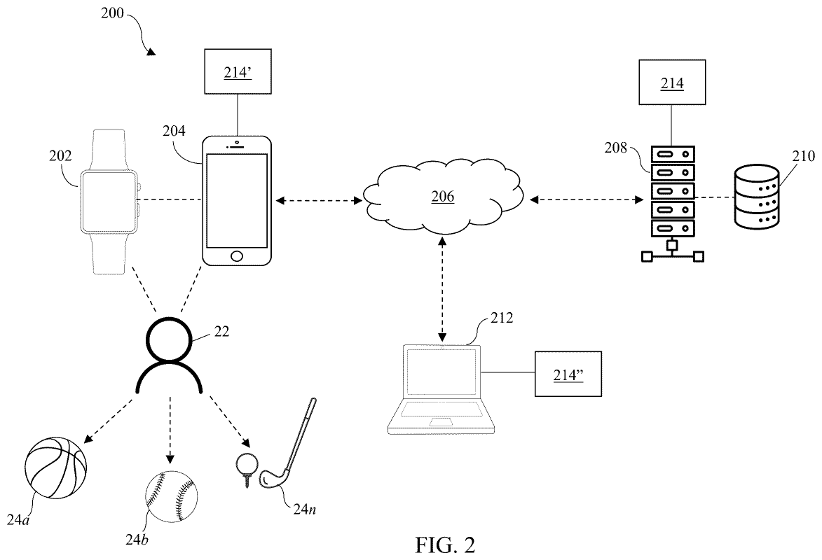

Referring now to FIG. 2, an architecture diagram of a sports biomechanics training system 200 is shown. In accordance with certain aspects of the present disclosure, system 200 may comprise a wearable sensor device 202, a mobile computing device 204, a client device 212 and an application server 208 communicably engaged with an application database. Wearable sensor device 202 may comprise a smart watch, such as an APPLE WATCH, a wearable activity tracker, such as a FITBIT, or other body-worn sensor devices. In accordance with certain aspects of the present disclosure, wearable sensor device 202 comprises one or more sensors configured to measure 3-axes of motion when worn by a user 22. In certain embodiments, the one or more sensors may comprise a three-axis accelerometer, e-compass, inertial measurement unit, gyro, magnetometer and the like. Mobile computing device 204 may comprise a smart phone or tablet computer. In accordance with certain aspects of the present disclosure, mobile computing device 204 may be communicably engaged with wearable sensor device 202 via a wireless data transfer interface (e.g., BLUETOOTH). In accordance with certain aspects of the present disclosure, mobile computing device 204 and client device 212 may be communicably engaged with application server 208 via a network communications interface 206. In accordance with certain aspects of the present disclosure, application server 208 may host a biomechanics training application 214. End user application 214 may comprise a plurality of encoded operations for a sports biomechanics training method. Mobile computing device 204 may be configured to execute a mobile application instance 214′ of biomechanics training application 214. Client device 212 may be configured to execute a web application instance 214″ of biomechanics training application 214.

In accordance with an exemplary use case of system 200, user 22 may launch mobile application instance 214′ at mobile computing device 204 and establish a wireless data transfer interface with wearable sensor device 202. In certain embodiments, wearable sensor device 202 may be configured to execute a native instance of biomechanics training application 214. User 22 may engage with one or more user interface elements of mobile application instance 214′ to select or configured at least one operational mode of mobile application instance 214′. In accordance with certain aspects of the present disclosure, the at least one operational mode comprises one or more algorithms associated with one or more specifications for at least one biomechanical action associated with one or more sport techniques. In certain embodiments, the at least one biomechanical action may comprise shooting a basketball 24a, throwing a baseball 24b, and swinging a golfclub 24n. Numerous additional biomechanical actions associated with additional sports are anticipated including, but not limited to, swinging a baseball bat, throwing a football, use of a lacrosse stick, use of a bow for archery, swinging a tennis racquet, among others. In accordance with certain aspects of the present disclosure, user 22 executes one or more instances of the specified biomechanical action (e.g., takes 20 free throw shots with a basketball). Wearable sensor device 202 is configured to measure the movements of user 22 in real-time. For example, if user 22 wears wearable sensor device 202 on the wrist of the user's non-shooting hand, then wearable sensor device 202 is configured to measure the real-time movement of the user's wrist while executing the specified biomechanical action. In accordance with certain aspects of the present disclosure, wearable sensor device 202 is configured to provide sensor input data to mobile computing device 204 in real-time. Mobile computing device 204 is configured to process the sensor input data according to one or more operations of mobile application instance 214′. In accordance with certain embodiments, the one or more operations of mobile application instance 214′ comprise operations for processing the sensor input data according to one or more algorithms to determine a degree of conformity between the movements of the user and a target or expected movement of the user. For example, at least one algorithm embodied by the one or more operations of mobile application instance 214′ may comprise an algorithm for analyzing g-force measurements of the user's non-shooting wrist along the x-, y-, or z-axis to determine if the g-force measurements exceed a predetermined threshold (for example, 2 g) in any direction. In accordance with certain embodiments, at least a portion of the one or more operations may be executed at a processor of wearable sensor device 202. Mobile application instance 214′ may be further configured to execute one or more operations to provide an output to the user in response to processing the sensor input data. In certain embodiments, the output may comprise an audible output (e.g., a tone or beep), a haptic output (e.g., a vibration), and/or a visible output (e.g., a flashing light). Mobile application instance 214′ may be further configured to execute one or more operations to store the user activity data (e.g., user-generated inputs and sensor input data) locally on a non-transitory storage medium of mobile computing device 204 and/or communicate the user activity data to application server 208 via network interface 206. Application server 208 may be configured to store the user activity data within application database 210. In accordance with certain embodiments, application server 208 may be configured to process the user activity data according to one or more operations of biomechanics training application 214 to analyze the user activity data (e.g., according to one or more algorithms) and generate one or more training recommendations for the user to improve his or her biomechanics for the specified sport. In certain embodiments, the one or more operations of biomechanics training application 214 may further comprise analyzing current session data with prior session data stored in application database 210 to generate one or more progress reports. In certain embodiments, biomechanics training application 214 may comprise a machine learning framework configured to provide one or more predictive training goals, training programs, and/or insights to improve the user's biomechanics.

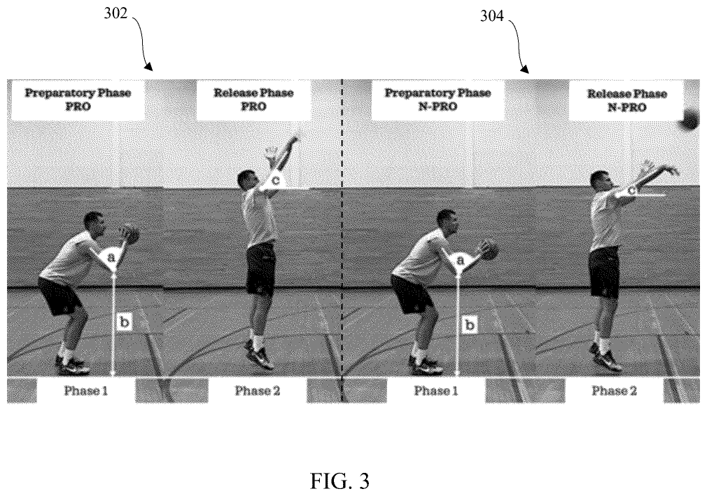

Referring now to FIG. 3, a functional diagram illustrating certain biomechanics for shooting a basketball is shown. As shown in FIG. 3, the desired/correct biomechanics for shooting a basketball are shown in illustration sequence 302. In accordance with certain aspects of the present disclosure, one or more algorithms of a biomechanics training application (e.g., biomechanics training application 214 of FIG. 2) may be configured to analyze one or more variables associated with the shooters body position and/or body movements. For example, the angle of the elbow in the preparatory phase, position of the arms relative to the body and/or the floor in the preparatory phase, the angle of the arm relative to the body in the release phase, the position and movement of the wrist of the non-shooting hand in the release phase, the position and movement of the wrist of the shooting hand in the release phase, among other biomechanical variables. Illustration sequence 304 illustrates undesired/incorrect biomechanics for shooting a basketball. In accordance with certain aspects of the present disclosure, the sensor input data generated when a user executes a biomechanically incorrect movement is different than (i.e., deviates from) the sensor input data that is generated when a user executes a biomechanically correct movement. In accordance with certain aspects of the present disclosure, the biomechanics training system is configured to identify and analyze such differences and provide the user with real-time feedback, statistics, and training recommendations.

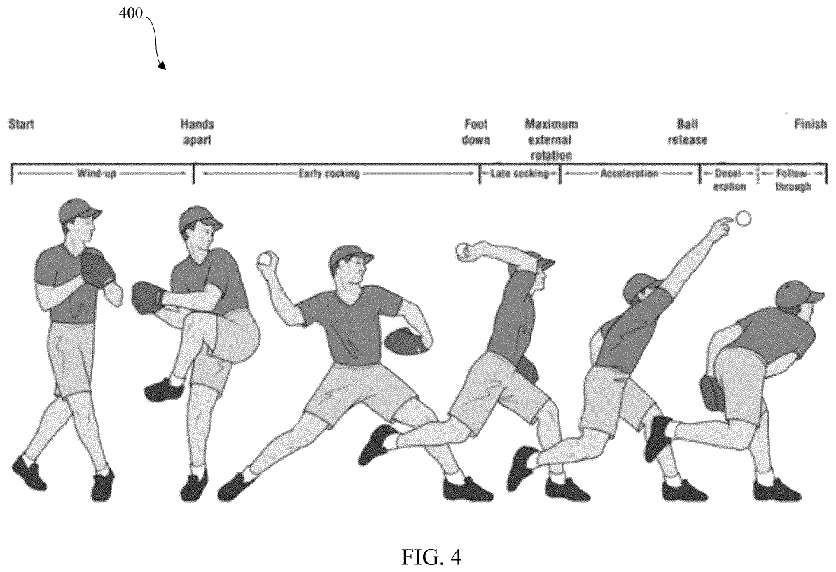

Referring now to FIG. 4, a functional diagram 400 illustrating certain biomechanics of throwing a baseball is shown. Diagram 400 illustrates the various biomechanical actions associated with a correct or desirable form for throwing a baseball. These actions may include various biomechanical phases including, for example, wind-up, early cocking, late cocking, acceleration, deceleration, and follow through. In accordance with certain aspects of the present disclosure, a user may wear one or more wearable sensor devices on the user's wrist of the throwing arm, wrist of the catching arm, torso, legs, head, or other location on the body to track and measure the user's movements for each of the biomechanical phases of the throwing a baseball.

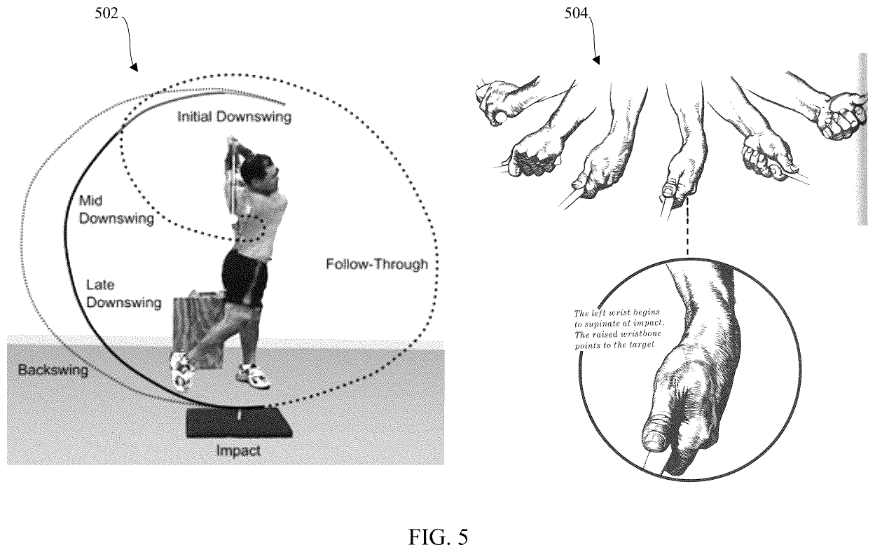

Referring now to FIG. 5, a functional diagram 500 illustrating certain biomechanics of swinging a golfclub is shown. Diagram 502 illustrates the various biomechanical actions associated with a correct or desirable form for swinging a golfclub. These actions may include various biomechanical phases including, for example, backswing, initial downswing, mid-downswing, late downswing, impact and follow through. Diagram 504 further illustrates certain biomechanics of the player's wrist in each of the biomechanical phases shown in diagram 502. In accordance with certain aspects of the present disclosure, a user may wear one or more wearable sensor devices on the user's right or left wrist (or both wrists), torso, one or both legs, one or both ankles, head, or other location on the body to track and measure the user's movements during each of the biomechanical phases of swinging a golfclub.

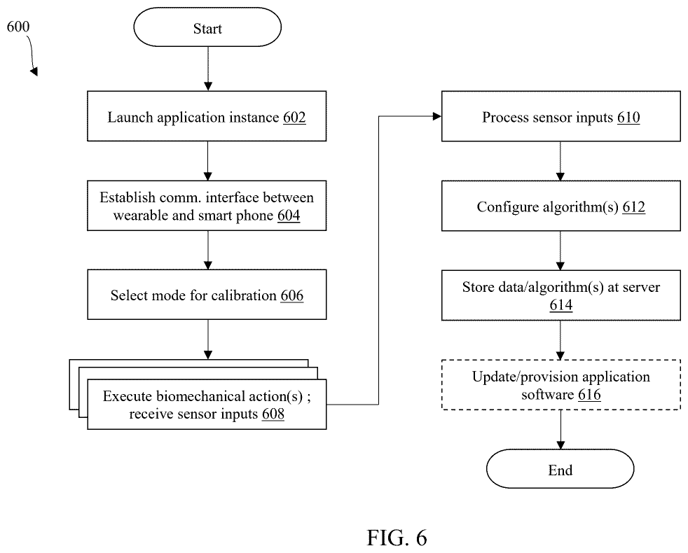

Referring now to FIG. 6, a process flow diagram of a routine 600 for a sports biomechanics training method is shown. The operations in routine 600 may be performed in the order presented, in a different order, or simultaneously. Further, in some exemplary embodiments, some of the operations may be omitted, added, modified, skipped, or the like without departing from the scope of the invention. Routine 600 may be embodied within one or more system operations of system 200, as shown in FIG. 2. In accordance with certain aspects of the present disclosure, routine 600 may comprise one or more steps 602-616 for calibrating one or more biomechanical sensor input values and/or configuring one or more algorithms for analyzing biomechanical sensor input data.

In accordance with certain embodiments, routing 600 may comprise one or more steps or operations for launching an instance of a sports biomechanics training application (e.g., at a mobile computing device of an end user) (Step 602). Routine 600 may further comprise one or more steps or operations for establishing a wireless communications interface (e.g., BLUETOOTH) between a wearable sensor device and a smart phone (Step 604). In certain embodiments, the wearable sensor device may comprise a smart watch. In certain embodiments, the system may comprise multiple wearable sensors that are attached to one or more locations of the user's body. In accordance with certain aspects of the present disclosure, routine 600 may proceed by executing one or more steps or operations for selecting (e.g., in response to a user-generated input at a user interface of the instance of the biomechanics training application) a mode for calibration of one or more biomechanical sensor input values (Step 606). The mode for calibration may be associated with one or more biomechanical sporting techniques including, but not limited to, shooting a basketball, throwing a baseball, and swinging a golfclub. The user may then execute the specified biomechanical actions and routine 600 may proceed by executing one or more steps or operations for receiving sensor inputs from the wearable sensors device(s) (Step 608). Routine 610 may proceed by executing one or more steps or operations for processing the sensors inputs (Step 610) and configuring one or more algorithms comprising a plurality of variables for the calibrated biomechanical action(s) (Step 612). Routine 614 may proceed by executing one or more steps or operations for storing the sensor data and the algorithms at an application server (Step 614) and may, optionally, comprise one or one or more steps or operations for updating an application logic of the biomechanics training application and/or provisioning one or more software update to an end user version of the biomechanics training application (Step 616). In accordance with certain aspects of the present disclosure, routine 600 enables a user to calibrate an algorithm of the biomechanics training application according to a customized set of biomechanical movements. For example, a basketball coach may wear the wearable sensor device in a calibration mode in order to calibrate the algorithm to match the specific shooting technique of the coach. A player may then wear the wearable sensor device in an operational mode of the biomechanics training application in order to receive sensor-driven feedback as to a degree to which the player's shooting technique conforms to the coach's shooting technique.

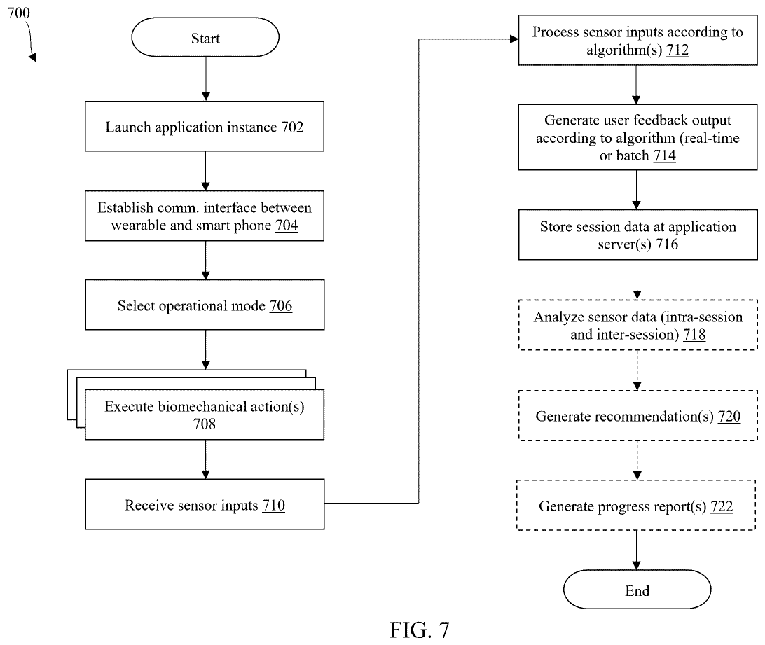

Referring now to FIG. 7, a process flow diagram of a routine 700 for a sports biomechanics training method is shown. The operations in routine 700 may be performed in the order presented, in a different order, or simultaneously. Further, in some exemplary embodiments, some of the operations may be omitted, added, modified, skipped, or the like without departing from the scope of the invention. Routine 700 may be embodied within one or more system operations of system 200, as shown in FIG. 2. Routine 700 may be successive or sequential to one or more steps or operations for routine 600, as shown in FIG. 6. In accordance with certain aspects of the present disclosure, routine 700 may comprise one or more steps 702-720 for executing an end user instance of a biomechanics training application to provide an end user with sensor-driven feedback related to a biomechanical technique for at least one sporting skill.

In accordance with certain aspects of the present disclosure, routine 700 may be initiated upon executing one or more steps or operations for launching an end user instance of a sports biomechanics training application (e.g., via an end user computing device) (Step 702) and establishing a wireless communications interface (e.g., BLUETOOTH) between a wearable sensor device and the end user computing device (e.g., a smart phone) (Step 704). Routine 700 may proceed by executing one or more steps or operations to enable a user to select an operational mode of the biomechanics training application (e.g., via one or more user-generated inputs at a graphical user interface) (Step 706). The operational mode may comprise a selected sport (e.g., basketball) and/or a selected biomechanical action for the selected sport (e.g., shooting free throws). The user may then execute one or more of specified biomechanical actions (Step 708) and routine 700 may execute one or more steps or operations for receiving one or more sensor inputs (e.g., via the wearable sensor device communicably engaged with the end user computing device) (Step 710). Routine 700 may proceed by processing the sensor inputs according to one or more algorithms of the biomechanical training application (Step 712). In accordance with certain embodiments, one or more steps or operations of Step 712 may be executed locally (e.g., via a processor of the wearable computing device and/or the end user computing device) and one or more steps or operations of Step 712 may be executed remotely (e.g., via a processor of a remote application server). Routine 700 may proceed by executing one or more steps or operations for generating a user feedback output according to a processing output of the one or more algorithms (Step 714). In certain embodiments, the user feedback output may be generated in real-time or may be batched at one or more intervals (e.g., at the end of the training session). In certain embodiments, a real-time output may include an audible output, a haptic output, and/or a visual output. Routine 700 may proceed by storing session data for the instance of the biomechanical training application at an application server (e.g., via an application database) (Step 716). Routine 700 may, optionally, comprise one or more steps or operations for analyzing (e.g., via a processor of the application server) intra-session data and/or inter-session data for the instance of the biomechanical training application (e.g., according to one or more data analytics framework) (Step 718). Routine 700 may further comprise one or more steps or operations for generating one or more training recommendations for the user (e.g., according to an output of Step 718) (Step 720) and/or generating one or more progress reports for the user to illustrate progress over time and predicted trends (Step 722).

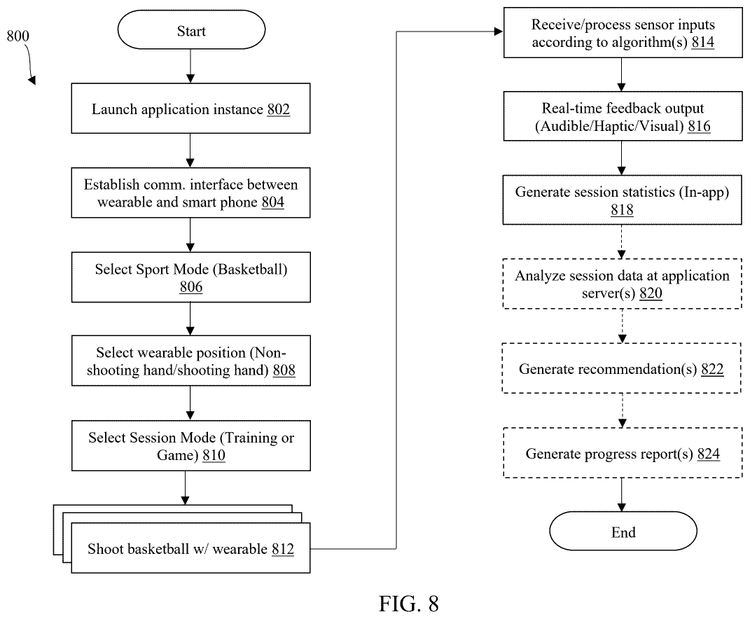

Referring now to FIG. 8, a process flow diagram of a routine 800 of a sports biomechanics training method is shown. The operations in routine 800 may be performed in the order presented, in a different order, or simultaneously. Further, in some exemplary embodiments, some of the operations may be omitted, added, modified, skipped, or the like without departing from the scope of the invention. Routine 800 may be embodied within one or more system operations of system 200, as shown in FIG. 2. Routine 800 may be successive or sequential to one or more steps or operations of routine 600, as shown in FIG. 6, and/or routine 700, as shown in FIG. 7. In accordance with certain aspects of the present disclosure, routine 800 may comprise one or more steps 802-824 for executing an end user instance of a biomechanics training application to provide an end user with sensor-driven feedback related to a biomechanical technique for at least one sporting skill.

In accordance with certain aspects of the present disclosure, routine 800 may be initiated upon executing one or more steps or operations for launching an end user instance of a biomechanics training application (e.g., via an end user computing device) (Step 802) and establishing a wireless communications interface (e.g., BLUETOOTH) between a wearable sensor device and the end user computing device (e.g., a smart phone) (Step 804). Routine 800 may proceed by executing one or more steps or operations to enable a user to select an operational mode of the biomechanics training application (e.g., via one or more user-generated inputs at a graphical user interface) (Step 806). In accordance with certain aspects of the present disclosure, the operational mode comprises a basketball mode. Routine 800 may proceed by executing one or more steps or operations to enable a user to select a position of the wearable sensor device (e.g., the shooting hand or the non-shooting hand) via one or more user-generated inputs at the graphical user interface (Step 808). Routine 800 may proceed by executing one or more steps or operations to enable a user to select a functional mode for the session (e.g., training mode or game mode) via one or more user-generated inputs at the graphical user interface (Step 810). In accordance with certain aspects of the present disclosure, the user executes one or more repetitions of shooting a basketball while wearing the wearable sensor device in communication with the end user computing device (Step 812). Routine 800 may proceed by receiving and processing a plurality of sensor input data (e.g., with at least one processor of the wearable sensor device and/or the end user computing device) according to one or more algorithms encoded in one or more operations of the biomechanics training application (Step 814). In accordance with certain embodiments, routine 800 may comprise one or more steps or operations for generating a real-time feedback output to the end user (e.g., via the wearable sensor device or the end user computing device) in response to an output of Step 814 (Step 816). In certain embodiments, the real-time feedback output may comprise a haptic output, a visual output, and/or an audible output. In accordance with certain aspects of the present disclosure, routine 800 may comprise one or more steps or operations for generating one or more session statistics based on the sensor input data and rendering one or more data visualizations at the graphical user interface of the end user application (Step 818). In accordance with certain aspects of the present disclosure, routine 800 may comprise one or more steps or operations for analyzing the session data at an application server (e.g., according to one or more data analytics framework) (Step 820) in order to generate one or more recommendations for the user to improve his or her basketball shooting technique (Step 822) and/or generate one or more progress reports to illustrate improvement in the user's basketball shooting technique over a specified time period (Step 824).

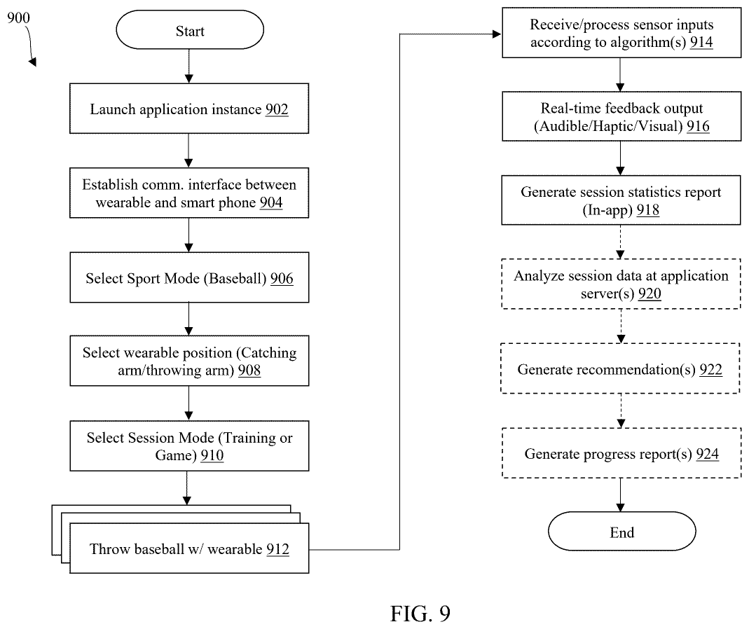

Referring now to FIG. 9, a process flow diagram of a routine 900 of a sports biomechanics training method is shown. The operations in routine 900 may be performed in the order presented, in a different order, or simultaneously. Further, in some exemplary embodiments, some of the operations may be omitted, added, modified, skipped, or the like without departing from the scope of the invention. Routine 900 may be embodied within one or more system operations of system 200, as shown in FIG. 2. Routine 900 may be successive or sequential to one or more steps or operations of routine 600, as shown in FIG. 6, and/or routine 700, as shown in FIG. 7. In accordance with certain aspects of the present disclosure, routine 900 may comprise one or more steps 902-924 for executing an end user instance of a biomechanics training application to provide an end user with sensor-driven feedback related to a biomechanical technique for at least one sporting skill.

In accordance with certain aspects of the present disclosure, routine 900 may be initiated upon executing one or more steps or operations for launching an end user instance of a biomechanics training application (e.g., via an end user computing device) (Step 902) and establishing a wireless communications interface (e.g., BLUETOOTH) between a wearable sensor device and the end user computing device (e.g., a smart phone) (Step 904). Routine 900 may proceed by executing one or more steps or operations to enable a user to select an operational mode of the biomechanics training application (e.g., via one or more user-generated inputs at a graphical user interface) (Step 906). In accordance with certain aspects of the present disclosure, the operational mode comprises a baseball mode. Routine 900 may proceed by executing one or more steps or operations to enable a user to select a position of the wearable sensor device (e.g., the throwing hand or the catching hand) via one or more user-generated inputs at the graphical user interface (Step 908). Routine 900 may proceed by executing one or more steps or operations to enable a user to select a functional mode for the session (e.g., training mode or game mode) via one or more user-generated inputs at the graphical user interface (Step 910). In accordance with certain aspects of the present disclosure, the user executes one or more repetitions of throwing a baseball while wearing the wearable sensor device in communication with the end user computing device (Step 912). Routine 900 may proceed by receiving and processing a plurality of sensor input data (e.g., with at least one processor of the wearable sensor device and/or the end user computing device) according to one or more algorithms encoded in one or more operations of the biomechanics training application (Step 914). In accordance with certain embodiments, routine 900 may comprise one or more steps or operations for generating a real-time feedback output to the end user (e.g., via the wearable sensor device or the end user computing device) in response to an output of Step 914 (Step 916). In certain embodiments, the real-time feedback output may comprise a haptic output, a visual output, and/or an audible output. In accordance with certain aspects of the present disclosure, routine 900 may comprise one or more steps or operations for generating one or more session statistics based on the sensor input data and rendering one or more data visualizations at the graphical user interface of the end user application (Step 918). In accordance with certain aspects of the present disclosure, routine 900 may comprise one or more steps or operations for analyzing the session data at an application server (e.g., according to one or more data analytics framework) (Step 920) in order to generate one or more recommendations for the user to improve his or her baseball throwing technique (Step 922) and/or generate one or more progress reports to illustrate improvement in the user's baseball throwing technique over a specified time period (Step 924).

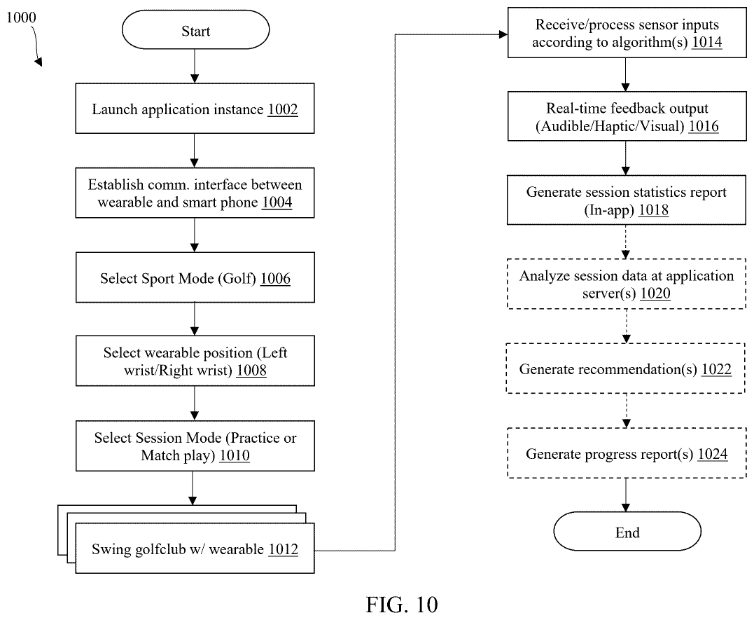

Referring now to FIG. 10, a process flow diagram of a routine 1000 of a sports biomechanics training method is shown. The operations in routine 1000 may be performed in the order presented, in a different order, or simultaneously. Further, in some exemplary embodiments, some of the operations may be omitted, added, modified, skipped, or the like without departing from the scope of the invention. Routine 1000 may be embodied within one or more system operations of system 200, as shown in FIG. 2. Routine 1000 may be successive or sequential to one or more steps or operations of routine 600, as shown in FIG. 6, and/or routine 700, as shown in FIG. 7. In accordance with certain aspects of the present disclosure, routine 1000 may comprise one or more steps 1002-1024 for executing an end user instance of a biomechanics training application to provide an end user with sensor-driven feedback related to a biomechanical technique for at least one sporting skill.

In accordance with certain aspects of the present disclosure, routine 1000 may be initiated upon executing one or more steps or operations for launching an end user instance of a biomechanics training application (e.g., via an end user computing device) (Step 1002) and establishing a wireless communications interface (e.g., BLUETOOTH) between a wearable sensor device and the end user computing device (e.g., a smart phone) (Step 1004). Routine 1000 may proceed by executing one or more steps or operations to enable a user to select an operational mode of the biomechanics training application (e.g., via one or more user-generated inputs at a graphical user interface) (Step 1006). In accordance with certain aspects of the present disclosure, the operational mode comprises a golf mode. Routine 1000 may proceed by executing one or more steps or operations to enable a user to select a position of the wearable sensor device (e.g., the right wrist or the left wrist) via one or more user-generated inputs at the graphical user interface (Step 1008). Routine 1000 may proceed by executing one or more steps or operations to enable a user to select a functional mode for the session (e.g., training mode or game mode) via one or more user-generated inputs at the graphical user interface (Step 1010). In accordance with certain aspects of the present disclosure, the user executes one or more repetitions of swinging a golfclub while wearing the wearable sensor device in communication with the end user computing device (Step 1012). Routine 1000 may proceed by receiving and processing a plurality of sensor input data (e.g., with at least one processor of the wearable sensor device and/or the end user computing device) according to one or more algorithms encoded in one or more operations of the biomechanics training application (Step 1014). In accordance with certain embodiments, routine 1000 may comprise one or more steps or operations for generating a real-time feedback output to the end user (e.g., via the wearable sensor device or the end user computing device) in response to an output of Step 1014 (Step 1016). In certain embodiments, the real-time feedback output may comprise a haptic output, a visual output, and/or an audible output. In accordance with certain aspects of the present disclosure, routine 1000 may comprise one or more steps or operations for generating one or more session statistics based on the sensor input data and rendering one or more data visualizations at the graphical user interface of the end user application (Step 1018). In accordance with certain aspects of the present disclosure, routine 1000 may comprise one or more steps or operations for analyzing the session data at an application server (e.g., according to one or more data analytics framework) (Step 1020) in order to generate one or more recommendations for the user to improve his or her golf swing technique (Step 1022) and/or generate one or more progress reports to illustrate improvement in the user's golf swing technique over a specified time period (Step 1024).

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational phases to be performed on the computer or other programmable apparatus to produce a computer-implemented process such that the code portions which execute on the computer or other programmable apparatus provide phases for implementing the functions/acts specified in the flowchart and/or block diagram block(s). Alternatively, computer program implemented phases or acts may be combined with operator or human implemented phases or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A method comprising:
providing a wireless communications interface between a wearable sensor device and at least one computing device, wherein the wearable sensor device is worn by a user;
launching an instance of an end user application on the at least one computing device;
selecting, via a user interface of the end user application, at least one operational mode for the end user application, wherein the at least one operational mode is associated with a specified biomechanical action of the user,
wherein the specified biomechanical action is associated with at least one sport,
receiving, with the wearable sensor device, one or more sensor inputs in response to the user performing one or more biomechanical actions;
processing, according to one or more operations of the end user application, the one or more sensor inputs; and
providing, with the wearable sensor device or the at least one computing device, one or more real-time feedback outputs to the user in response to processing the one or more sensor inputs.
